(19) United States
(12) Patent Application Publication
(10) Pub. No.: US 2023/0285607 A1
(43) Pub. Date: Sep. 14, 2023

(54) ULTRAVIOLET LIGHT EMITTING URINARY CATHETER

(71) Applicant: Tomasz Dziura, Wheeling, IL (US)

(72) Inventor: Tomasz Dziura, Wheeling, IL (US)

(21) Appl. No.: 17/692,842

(22) Filed: Mar. 11, 2022

(51) Int. Cl. A61L 2/10; H05B 47/19; F21S 4/26; A61M 25/00

(52) U.S. Cl. CPC A61L 2/10; H05B 47/19; F21S 4/26; A61M 25/0026; A61M 2025/0019; F21Y 2115/10

(57) ABSTRACT

A disinfecting catheter includes an elongated body having a plurality of lumens. The elongated body includes an insertion end having one or more openings in a sidewall, and a second end is a multifurcated end. A retaining balloon is arranged around an outer portion of the insertion end of the elongated body. An electrode is arranged at least partially within the multifurcated end of the elongated body. A plurality of ultraviolet (UV) light emitting diodes are arranged along the elongated body and connected to the electrode. A UV light controller controls the intensity and duration of the UV light output by the UV light emitting diodes. The UV light controller can receive commands from a smartphone to operate/adjust the UV light emitting diodes.

### BACKGROUND

### Technical Field

The present disclosure generally relates to ultraviolet cleaning devices, and more particularly, to an ultraviolet light emitting urinary catheter device.

### Description of the Related Art

With advances in treatment of a number of illnesses that are extending the average lifespan, there is an increase in the use of urinary catheters. For example, a urinary catheter such as a Foley catheter is designed to be inserted in the urethra, with one end in the bladder, and another end in a collection bag. On the bladder end of the catheter, a balloon (e.g. typically axially-arranged adjacent a center portion of the catheter) is inflated with 5cc to 10cc of water, so that the catheter does not slip out of the bladder until the balloon is deflated. Urine flows from the bladder through a channel portion of the catheter and exits into a container, often a collection bag that is emptied at least once a day, and typically changed every few days. Some Foley catheters are also used as a suprapubic catheter. Instead of the catheter being inserted into the urethra, the catheter is arranged external to the body and enters the bladder via a stoma initially made by a surgeon in a portion of the skin above the public area. A collection bag is attached to another end of the catheter. The collection bag is emptied daily, and replaced at least twice per week.

For most patients, the catheter is changed every 3-4 weeks by a nurse and/or a urologist. The use of catheters tend to increase the incidence of urinary tract infections (UTIs), which can be difficult to control, and can spread to the kidneys. Medicines such as methenamine Hippurate is often prescribed to pre-empt such UTIs. The UTIs tend to create particulates in the urine that can cause the catheter to clog, requiring prompt replacement, which may have to be performed at a hospital emergency room outside of regular hours and on the weekends. Accordingly, infection control associated with the use of catheters continues to be an area of research.

### SUMMARY

In one embodiment, A disinfecting catheter includes an elongated body having a plurality of lumens. The elongated body includes an insertion end having one or more openings in a sidewall, and a second end is a multifurcated end. A retaining balloon arranged around an outer portion of the insertion end of the elongated body. An electrode is arranged at least partially within the multifurcated end of the elongated body. A plurality of ultraviolet (UV) light emitting diodes are arranged along the elongated body and connected to the electrode.

In an embodiment, a UV light controller electrically connected to the plurality of UV light emitting diodes via the electrode.

In an embodiment, the plurality of UV light emitting diodes is arranged in one of the plurality of lumens in the elongated body, and

In an embodiment, the UV light emitting diodes emits UV light having a wavelength between 200 and 280 nm.

In an embodiment, the plurality of lumens total three lumens arranged in the elongated body and the multifurcated end comprises a trifurcated end. A first lumen is arranged in fluid communication with the retaining balloon, a second lumen is configured to transport fluid from a bladder. A third lumen has the plurality of UV light emitting diodes arranged therein that are electrically connected to the UV controller.

In an embodiment, a UV light reflective coating is arranged along an inner surface of the elongated body.

In an embodiment, a UV light absorbing coating is arranged along an inner surface of the elongated body.

In an embodiment, the elongated body is constructed of a material that substantially blocks UV light between 200-300 nm, and at least one lumen is constructed of silicone.

In an embodiment, the UV light controller is arranged external to the disinfecting catheter.

In an embodiment, the UV light controller is configured to actuate the UV light emitting diodes in a least an intensity and/or a duration of time for a disinfection cycle.

In an embodiment, the UV light controller includes a wireless receiver to receives commands via at least one wireless technology selected from the group consisting of Wi-Fi, Bluetooth, WiBRO, WiMAX, and Near-Field Communications (NFC).

In an embodiment, the UV controller includes a wireless receiver that receives commands from a smart device application.

In an embodiment, the UV controller is configured for connection to a catheter collection bag.

In an embodiment, the third lumen is longer than the first lumen and the second lumen and extends from the elongated body for attachment of the UV controller to a catheter collection bag.

In an embodiment, the plurality of UV light emitting diodes is connected to the UV controller by a wire that extends to the catheter collection bag.

In an embodiment, an additional UV light emitting diode is arranged to output UV light toward fluid in the catheter collection bag.

In one embodiment, a disinfecting catheter includes an elongated body having a proximal end configured for insertion into a bladder, and a distal end configured for connection to a collection bag. The elongated body has a plurality of lumens arranged therein constructed of a material that substantially permits passage of UVC light, and at least one opening at a tip of the proximal end in fluid communication with a first lumen. A retaining balloon along an exterior of the proximal end of the elongated body is arranged in fluid communication with a second lumen. A plurality of ultraviolent (UV) light emitting diodes arranged at predetermined distances in the elongated body and emit UV-C light.

In an embodiment, a UV light controller configured to control operation of the light emitting diodes is electrically connected to the plurality of UV light emitting diodes, the UV controller is arranged at least partially external from the distal end of the elongated body.

In an embodiment, a third lumen is arranged in the elongated body, the third lumen houses the plurality of UV light emitting diodes.

In an embodiment, the UV light controller is configured to adjust an intensity and/or a duration of the UV light emitted from the UV light emitting diodes.

In an embodiment, the UV light controller receives commands from a smartphone to adjust an intensity and/or a duration of the UV light emitted from the UV light emitting diodes.

These and other features will become apparent from the following detailed description of illustrative embodiments thereof, which is to be read in connection with the accompanying drawings.

### BRIEF DESCRIPTION OF THE DRAWINGS

The drawings are of illustrative embodiments. They do not illustrate all embodiments. Other embodiments may be used in addition to or instead. Details that may be apparent or unnecessary may be omitted to save space or for more effective illustration. Some embodiments may be practiced with additional components or steps and/or without all the components or operations that are illustrated. When the same numeral appears in different drawings, it refers to the same or like components or operations.

FIG. 1 illustrates a perspective view of a disinfecting catheter according to an illustrative embodiment of the present disclosure.

FIG. 2 illustrates a view from a trifurcated end of the disinfecting catheter according to an illustrative embodiment of the present disclosure.

FIG. 3 is illustrative example of a type of collection bag used in conjunction with a disinfecting catheter according to an illustrative embodiment of the present disclosure.

FIG. 4 is a block diagram of a smart device in communication with a UV controller according to an illustrative embodiment of the present disclosure.

### DETAILED DESCRIPTION

### Overview

In the following detailed description, numerous specific details are set forth by way of examples to provide a thorough understanding of the relevant teachings. However, it should be understood that the present teachings may be practiced without such details. In other instances, well-known methods, procedures, components, and/or circuitry have been described at a relatively high level, without detail, to avoid unnecessarily obscuring aspects of the present teachings.

As used herein, the term “catheter” is not limited to a particular type of catheter. For example, while the drawings show a Foley Catheter, the claimed subject matter is applicable to other types of catheters used for evacuation from a bladder. In addition, the claimed subject matter is applicable to catheters used in other parts of a living body (e.g. veins). Although the retaining balloon is shown inflated with fluid, the claimed subject matter is not limited thereto, and air and/or a gas may be used in place of fluid.

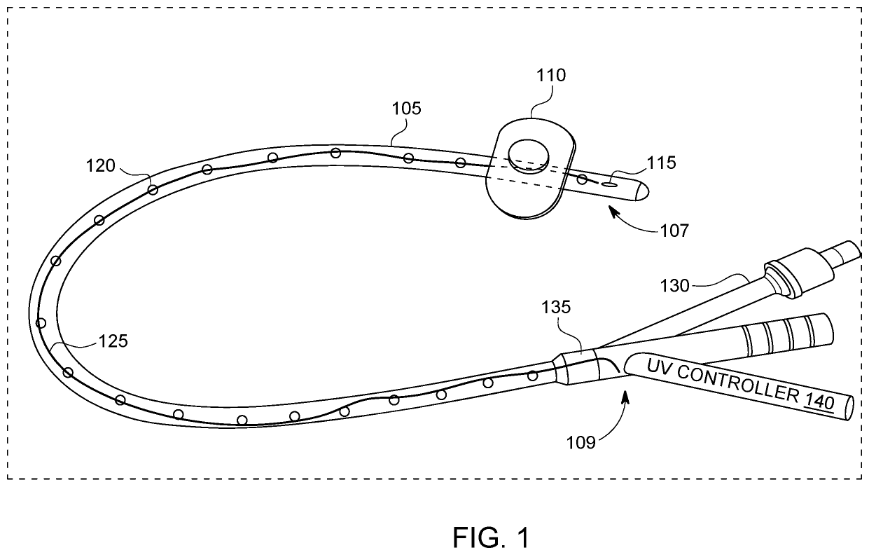

FIG. 1 illustrates a perspective view 100 of a disinfecting catheter according to an illustrative embodiment.

As shown in FIG. 1, a catheter 105 has an elongated body that includes an insertion end 107, and a multifurcated end 109. The insertion end 107 includes a tip having one or more holes 115 in a wall of the elongated body and a retaining balloon 110 that is inflated inside the bladder via one lumen 130 (e.g. an inflation lumen) at the multifurcated end. The retaining balloon 110 is arranged substantially around a portion of an exterior of the elongated body. While typically inflated with fluid, I is possible to inflate with a gas, taking into account volumetric changes based on variants in temperature.

The catheter 105 shown is known as a “foley catheter” with a size of 20 French, a balloon size of 5cc. Medical personnel often overinflate the retaining balloon of 5cc with 10cc of fluid to ensure a tight fit around the opening of the bladder. The particular catheter 105 shown is often used for a suprapubic insertion through a stoma. However, Foley catheters of the type shown and described herein are also inserted through the urethra (typically in a smaller size) and the retaining balloon 110 is inflated in a similar manner,

A plurality of UV light emitting diodes 120 are arranged in the catheter 105 at predetermined or random intervals. An electrode 125 such as a wire or conducting cable is connected to the UV light emitting diodes 120. Although the catheter in FIG. 1 shows an apparent series electrical connection of the UV light emitting diodes 120, a parallel electrical connection can be provided to increase the overall reliability of the operation of the catheter 105. There is also shown in FIG. 1 that one of the UV light emitting diodes is arranged to the right of the retaining balloon 110. This arrangement is optional, as the portion of the catheter 105 from the retaining balloon 110 to the tip is arranged in the bladder, and the inclusion of a UV light emitting diode in the portion of the catheter that is in the bladder may not be desirable in all cases. It is also to be understood that the size, arrangement and spacing of the UV light emitting diodes are provided for illustrative purposes and the claimed subject matter of the present disclosure is not limited to the arrangement shown in FIG. 1.

Still referring to FIG. 1, the multifurcated end shown can be referred to as a trifurcated end, as there are three branches that connect to respective lumens within the elongated body of the catheter. There is an inflation lumen 130 to inflate the retaining balloon 110. A UV controlling lumen 145 houses the UV light emitting diodes 120, and an electrode that may end from the UV controlling lumen 145 may be connected to the UV controller 140. An evacuation lumen 135 provides the path for fluid to be evacuated from the bladder, and is typically connected to a collection bag (not shown). For example, the collection bag may be a 500 ml or a 1000 ml collection bag.

It is to be understood that although the above description teaches that the UV light emitting diodes are arranged in one of the lumens, the claimed subject matter is not so limited. For example, the plurality of UV light emitting diodes 120 may be arranged along in inner surface of the elongated body that houses the other lumens for inflation and evacuation, and thus constitutes a bifurcated end (as opposed to the trifurcated end shown in FIG. 1). Moreover, a fourth lumen (not shown) for flushing the catheter with sterile water without disconnecting the evacuation lumen 135 may also be provided to reduce the possibility of introducing bacteria into the catheter during the flushing process.

As UV light for germicidal purposes is typically UVC light in the 200-298 nm range, it is to be understood that other ranges may be used for particular types of disinfection. A common wavelength used for infection is 254 nm, but the catheter shown and described is not so limited. Currently, a 222 nm wavelength is being studied as potentially less harmful to skin and eyes.

As there is a potential for UV light in the UVC range to cause damage to tissue (such as in the urethra) of the bladder, or possibly to skin in the case of a suprapubic connection, the elongated body of the catheter 105 may be coated with a UV light blocking material, or constructed of a UV light blocking material. However, the lumens are constructed of a material that permits passage of UV light to perform disinfection. For example, UV light passes from the UV controlling lumen 145 to the evacuation lumen 135 to disinfect the path of fluid evacuation from the bladder, while the elongated body of the catheter 105 housing the lumens blocks the passage of UV light from exiting the catheter to protect the patient from any harmful effects. In the event that the UV catheter is not coated with or constructed from a UV protective material, a physician's discretion may be used to weigh the risk vs benefit in the particular patient's case. Most jurisdictions require a physician's prescription for the purchase of a conventional catheter, and the selection of a UV disinfecting catheter from a variety of types would include consideration of the UV type and amount, as well as whether a UV protective coating or construction is desired may be part of the physician's discretion as to the particular prescribed catheter.

A reflective material may be layer onto the UV light blocking material of the elongated body. In such construction, the UV light emitted from the UV light emitting diodes 120 will pass through the evacuation lumen 135 and reflect off the surfaced of the elongated body and be directed back into evacuation lumen 135 to enhance a disinfection process.

The UV controller 140 is shown being adjacent the branches of the catheter. However, the electrode 125 may be of sufficient length so that the UV controller is attachable to the collection bag, for example, with Velcro™. The UV controller 140 may be reusable, and can be connected to the electrode via, for example, a connector or plug/receptacle.

The UV controller 140 is configured to turn on/off the UV light emitting diodes for a cleaning cycle. The UV controller may have a consumer-supplied battery, or a have a battery included for operation. In the case of a catheter being used for evacuation from a bladder, typically the catheter is discarded within a month or less. Thus, in the case of a single use UV controller, a relatively inexpensive battery with a short life can be provided by a consumer, or included in the construction. It is also possible to use a more durable battery such as a lithium ion to power the UV controller.

In addition to being turned on or off, the UV controller may control one or both an intensity and/or duration of the operation of the UV light emitting diodes 120 for a particular disinfection cycle. A receiver, or a transmitter may be included in the UV controller, and instructions for an individualized disinfection cycle can be sent to the UV controller via a smartphone using Wi-Fi, Bluetooth, near-field communication, Wibro, WiMAX, etc. For example, if a patient's urine culture indicates the presence of a particular bacteria, a doctor or medical personnel can prescribe an increased duration and/or intensity of a UV light to be transmitted. There may also be a table in a smartphone application or in a server that provides individualized instructions to the UV controller regarding a disinfection cycle that maximizes disinfection. As UV light can break down polymers and other materials used in the fabrication of a catheter, the ability to vary the intensity and/or duration of the disinfection operations may maximize catheter life and enhance safety provided to a patient.

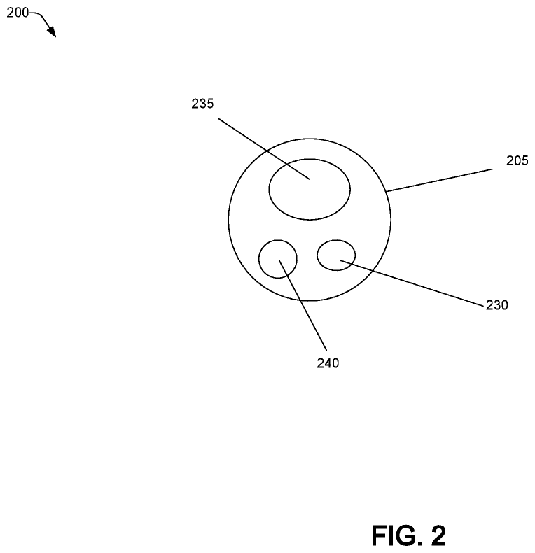

FIG. 2 illustrates a view 200 from a trifurcated end of the disinfecting catheter according to an illustrative embodiment of the present disclosure. As shown in FIG. 2, the elongated body 205 has a plurality of lumens housed therein. The evacuating lumen 235 has the largest diameter. The inflation lumen 230 and UV controlling lumen 245 are shown as being smaller in diameter than the evacuating lumen. However, the claimed subjected matter is not limited to the depiction of FIG. 2. The lumens may be equally sized in terms of diameters, or all of the lumens may have different diameters, or a lumen other than the evacuating lumen may be larger than the other lumens. It is also to be understood that there may be fewer lumens than shown. For example, the UV light emitting electrodes may be arranged along an inner surface of the elongated tube without being arranged in a lumen.

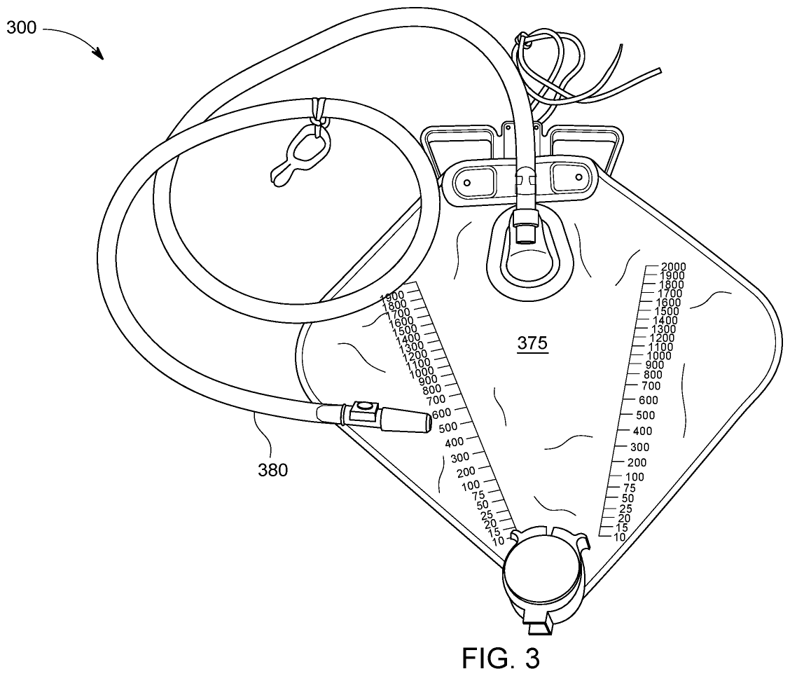

FIG. 3 shows an example 300 of a collection bag 375 that is connected to the disinfecting catheter according to an illustrative embodiment of the present disclosure. The collection bag 375 has a tube 380 which is typically inserted in the evacuation lumen (see FIG. 1). The collection bag may be embodied in many types of shapes and/or sizes than shown,

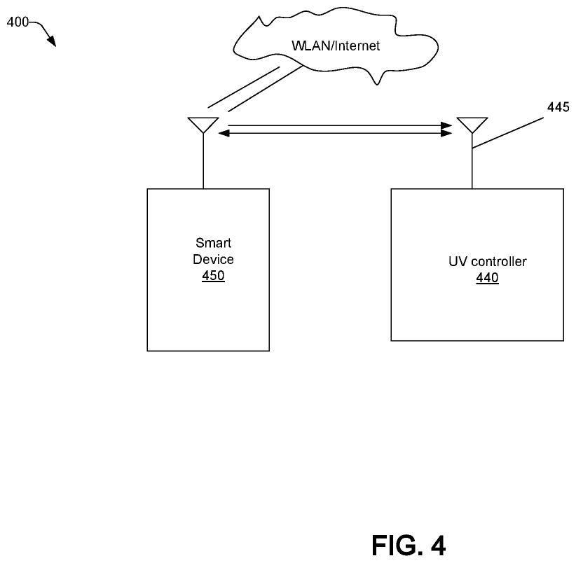

FIG. 4 is a block diagram of a smart device in communication with a UV controller according to an illustrative embodiment of the present disclosure. The UV controller 440 is shown with an antenna 445 that can receive instructions wirelessly from a smart device 450 such as a smartphone or table. The smart device 450 can directly communicate with the UV controller 440 via any of Wi-Fi, Bluetooth, NFC, Wibro, WiMAX, etc.,, and may receive instructions via an installed application, or through a server via a WLAN of cellular network. The instructions regarding intensity and/or duration of the UV light emitting diodes 12 (see FIG. 1) can be individualized for enhanced disinfection operations. The UV controller may have just a receiver, or a transmitter and receiver (or a transceiver). While the smart device is shown with a single antenna, it is understood by an artisan that multiple antennas and/or antenna arrays may be used to communicate with both the UV controller 445 and with a WLAN or a cellular network.

### Conclusion

The descriptions of the various embodiments of the disinfecting catheter according to present disclosure have been presented for purposes of illustration, but are not intended to be exhaustive or limited to the embodiments disclosed. Many modifications and variations will be apparent to those of ordinary skill in the art without departing from the scope and spirit of the described embodiments. For example, UVA or UVB light may be used with different effectiveness of disinfection. While the catheter shown and described may be constructed of silicone, or its lumens constructed of silicone, other UV transmissible materials can be used. In addition, the principles shown and described herein are not limited to a bladder, and can be used in veins and arteries for catheters used in conjunction with heart surgery, etc. The terminology used herein was chosen to best explain the principles of the embodiments, the practical application or technical improvement over technologies found in the marketplace, or to enable others of ordinary skill in the art to understand the embodiments disclosed herein.

The components, operations, steps, features, objects, benefits, and advantages that have been discussed herein are merely illustrative. None of them, nor the discussions relating to them, are intended to limit the scope of protection. While various advantages have been discussed herein, it will be understood that not all embodiments necessarily include all advantages. Unless otherwise stated, all measurements, values, ratings, positions, magnitudes, sizes, and other specifications that are set forth in this specification, including in the claims that follow, are approximate, not exact. They are intended to have a reasonable range that is consistent with the functions to which they relate and with what is customary in the art to which they pertain.

Numerous other embodiments of the disclosure are also contemplated. There are also embodiments of the disclosure in which the components and/or operations are arranged and/or ordered differently than described and shown herein.

It will be understood that the terms and expressions used herein have the ordinary meaning as is accorded to such terms and expressions with respect to their corresponding respective areas of inquiry and study except where specific meanings have otherwise been set forth herein. Relational terms such as first and second and the like may be used solely to distinguish one entity or action from another without necessarily requiring or implying any such actual relationship or order between such entities or actions. The terms “comprises,” “comprising,” or any other variation thereof, are intended to cover a non-exclusive inclusion, such that a process, method, article, or apparatus that comprises a list of elements does not include only those elements but may include other elements not expressly listed or inherent to such process, method, article, or apparatus. An element proceeded by “a” or “an” does not, without further constraints, preclude the existence of additional identical elements in the process, method, article, or apparatus that comprises the element.

What is claimed is:

1. A disinfecting catheter, comprising:
an elongated body having a plurality of lumens therein, the elongated body including a first end comprising an insertion end having one or more openings in a sidewall and a second end comprising a multifurcated end;
a retaining balloon arranged around an outer portion of the insertion end of the elongated body;
an electrode arranged at least partially within the multifurcated end of the elongated body;
a plurality of ultraviolet (UV) light emitting diodes arranged along the elongated body and connected to the electrode.

2. The disinfecting catheter according to claim 1, further comprising
a UV light controller electrically connected to the plurality of UV light emitting diodes via the electrode.

3. The disinfecting catheter according to claim 1,
wherein the plurality of UV light emitting diodes is arranged in one of the plurality of lumens in the elongated body, and
wherein the UV light emitting diodes emits UV light having a wavelength between 200 and 280 nm.

4. The disinfecting catheter according to claim 1,
wherein the plurality of lumens comprises three lumens arranged in the elongated body and the multifurcated end comprises a trifurcated end,
a first lumen is arranged in fluid communication with the retaining balloon;
a second lumen is configured to transport fluid from a bladder, and
a third lumen has the plurality of UV light emitting diodes arranged therein that are electrically connected to the UV controller.

5. The disinfecting catheter according to claim 4, further comprising a UV light reflective coating arranged along an inner surface of the elongated body.

6. The disinfecting catheter according to claim 4, further comprising a UV light absorbing coating arranged along an inner surface of the elongated body.

7. The disinfecting catheter according to claim 4, wherein the elongated body is constructed of a material that substantially blocks UV light between 200-300 nm, and at least one of the plurality of lumens is constructed of silicone.

8. The disinfecting catheter according to claim 4, wherein the UV light controller is arranged external to the disinfecting catheter.

9. The disinfecting catheter according to claim 4, wherein the UV light controller is configured to actuate the UV light emitting diodes in a least one or more of intensity and duration for a disinfection cycle.

10. The disinfecting catheter according to claim 4, wherein the UV light controller includes a wireless receiver to receives commands via at least one wireless technology selected from the group consisting of Wi-Fi, Bluetooth, WiBRO, WiMAX, and Near-Field Communications (NFC).

11. The disinfecting catheter according to claim 4, wherein the UV controller includes a wireless receiver that receives commands from a smart device application.

12. The disinfecting catheter according to claim 4, wherein the UV controller is configured for connection to a catheter collection bag.

13. The disinfecting catheter according to claim 4, wherein the third lumen is longer than the first lumen and the second lumen and extends from the elongated body for attachment of the UV controller to a catheter collection bag.

14. The disinfecting catheter according to claim 4, wherein the plurality of UV light emitting diodes are connected to the UV controller by a wire that extends to the catheter collection bag.

15. The disinfecting catheter according to claim 14, further comprising an additional UV light emitting diode configured to output UV light at fluid in the catheter collection bag.

16. A disinfecting catheter, comprising:
an elongated body having a proximal end configured for insertion into a bladder, and a distal end configured for connection with a collection bag;
the elongated body having a plurality of lumens arranged therein constructed of a material that substantially permits passage of UVC light, and at least one opening at a tip of the proximal end in fluid communication with a first lumen;
a retaining balloon in fluid communication with a second lumen of the plurality of lumens adjacent the proximal end of the elongated body; and
a plurality of ultraviolent (UV) light emitting diodes arranged at predetermined distances in the elongated body;
wherein the plurality of UV light emitting diodes emits UV-C light.

17. The disinfecting catheter according to claim 16, further comprising:
a UV light controller electrically connected to the plurality of UV light emitting diodes, the UV controller is arranged at least partially external to the distal end of the elongated body.

18. The disinfecting catheter according to claim 16, further comprising a third lumen arranged in the elongated body, the third lumen houses the plurality of UV light emitting diodes.

19. The disinfecting catheter according to claim 17, wherein the UV light controller is configured to adjust an intensity and/or a duration of the UV light emitted from the UV light emitting diodes.

20. The disinfecting catheter according to claim 17, wherein the UV light controller receives commands from a smartphone to adjust an intensity and/or a duration of the UV light emitted from the UV light emitting diodes.
